(19) United States
(12) Patent Application Publication
(10) Pub. No.: US 2023/0192601 A1
(43) Pub. Date: Jun. 22, 2023

(54) SCALED-UP SYNTHESIS OF LOMUSTINE UNDER CONTROL FLOW CONDITIONS

(71) Applicant: Purdue Research Foundation, West Lafayette, IN (US)

(72) Inventors: David Harley Thompson, West Lafayette, IN (US); Zoltan Kalman Nagy, West Lafayette, IN (US); Ahmed Mufti, Lafayette, IN (US); Jaron Mackey, Austin, TX (US)

(73) Assignee: Purdue Research Foundation, West Lafayette, IN (US)

(21) Appl. No.: 17/924,865

(22) PCT Filed: Mar. 9, 2021
(86) PCT No.: PCT/US2021/021429

§ 371 Date: Nov. 11, 2022

Related U.S. Application Data
(60) Provisional application No. 63/023,020, filed on May 11, 2020.

(51) Int. Cl. C07C 273/18

(52) U.S. Cl. CPC C07C 273/18

(57) ABSTRACT

The present disclosure provides processes and apparatuses for the scaled-up manufacture of lomustine via continuous flow manufacture. Such continuous flow processes may optionally include the crystallization of lomustine and the apparatuses may optionally include crystallization apparatuses/reactors in either batch or continuous flow design. In one aspect of the disclosure, a process for making lomustine is provided comprising treating solutions of 2-chloroethylisocyanate with a solution of cyclohexylamine with continuous-flow pumps in a gram-flow reactor to form a combined solution, adding deionized water with a continuous flow-pump to the combined solution to form a liquid-organic phase solution, extracting the organic phase from the solution and treating with a solution of t-butyl nitrite with a continuous flow pump in a gram flow reactor to form lomustine.

### GOVERNMENT SUPPORT

This invention was made with government support under CA023168 awarded by the National Institutes of Health, W911NF-16-2-0020 awarded by the Defense Advanced Research Projects Agency, and FD-U-006738 awarded by the US Food & Drug Administration. The government has certain rights in the invention.

### FIELD OF INVENTION

This disclosure provides novel methods of synthesizing lomustine drug in scalable size under continuous flow conditions.

### BACKGROUND

Lomustine, a widely used anticancer agent, is a highly lipophilic alkylating agent that produces chloroethyl carbonium ions and carbamylating intermediates in vivo. These electrophilic compounds attack the nucleophilic sites on DNA to form alkylated products. Other anticancer agents such as mitomycin C, streptonigrin, bleomycin, and the anthracyclines require bioactivation to react with their cellular targets, whereas lomustine does not require pre-activation. Unlike alkylating agents that form adducts at the most reactive N⁷ position of guanine, chloroethylating compounds like lomustine form adducts at O⁶, leading to interstrand DNA cross-linking. If DNA repair does not occur, this crosslinking can cause double strand breaks during DNA replication, eventually leading to cell death via apoptosis.

Lomustine, 1-(2-chloroethyl)-3 -cyclohexyl-l-nitroso-urea (commercial names: CCNU, CeeNU, Gleostine) is used as an oral antineoplastic agent that is administered every 6 weeks. It was first evaluated in clinical trials in the late 1960s and approved by the US FDA in 1976 for primary and metastatic brain tumors as well as Hodgkin's lymphoma. Bristol-Myers Squibb originally held the patent for the agent under the brand name CeeNu. In 2014, Next Source Biotechnology LLC (NSB) was approved by the FDA for the rebranding of lomustine under the trade name Gleostine. The average wholesale price for one dose of rebranded Gleostine is $1,645.68, while the generic formulation costs $203.38. The huge price discrepancy (>800%) between Gleostine and the generic formulation has created patient access problems, and created a need for lower-costing lomustine.

Continuous flow synthesis has been reported as an efficient methodology and has been explored in both industry and academic labs for the last few decades. Compared to traditional batch synthesis processes, flow reactors provide better control over reaction conditions and selectivity owing to rapid mixing and precise control of reaction parameters such as temperature, stoichiometry, pressure, and residence time. The enhanced heat and mass transfer capabilities also provide safer and greener operational conditions. Generally, these aspects of continuous flow synthesis contribute to improved chemical reaction efficiency and shorter reaction times, enabling process intensification, and more facile scale-up, with improved quality and consistency in production. Motivated by these factors, continuous flow synthesis of active pharmaceutical ingredients has recently become more attractive, however, efficient execution of multistep reactions in a telescoped manner still remains a challenge due to challenges arising from workup conditions, solvent switches, and flow rate differences. Moreover, optimization of continuous flow conditions and analysis require significant investments in time and material.

In 62/746,045 and Ser. No. 16/654,103 now published as US20200115330A1, we disclose novel methods for producing lomustine under continuous flow conditions. Herein we further describe novel methods of producing lomustine under continuous flow conditions which improve upon the process disclosed in 62/746,045 and Ser. No. 16/654,103 now published as US20200115330A1, the contents of which are part of this disclosure under Attachment A and are further hereby incorporated by reference in their entirety.

### SUMMARY

In one aspect of the disclosure, a process for making lomustine is provided comprising treating solutions of 2-chloroethylisocyanate with a solution of cyclohexylamine with continuous-flow pumps in a gram-flow reactor to form a combined solution, adding deionized water with a continuous flow-pump to the combined solution to form a liquid-organic phase solution, extracting the organic phase from the solution and treating with a solution of t-butyl nitrite with a continuous flow pump in a gram flow reactor to form lomustine.

In another aspect of the disclosure, an apparatus substantially the same as FIG. 2 is provided.

In an additional aspect of the disclosure, an apparatus substantially the same as FIG. 3 is provided.

### BRIEF DESCRIPTION OF THE DRAWINGS

FIG. 1 shows a diagram for making and crystallizing lomustine.

FIG. 2 shows a diagram for making and crystallizing lomustine

FIG. 3 shows a diagram for making and crystallizing lomustine.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

Unless defined otherwise, the scientific and technology nomenclatures have the same meaning as commonly understood by a person in the ordinary skill in the art pertaining to this disclosure.

Continuous-flow manufacturing at scale presents particular challenges. It is important to identify solvents where the reactants are sufficiently soluble so that throughput is maximized without precipitation that could force a shutdown. Another challenge in continuous-flow manufacturing is product purity. Crystallization is usually the best way to achieve high purity, but that is more difficult to achieve continuously and contamination in the mother liquor should be low to enable such crystallization.

With respect to lomustine, in order to adequately produce lomustine to meet commercial need, a flow-reactor should be able to produce on the order of 250 grams/day or more. The synthesis set forth in Attachment A is optimized to provide only on the order of about 110 mg/hour which translates into only about 1% of a scale-up need if running 24 hours per day. The disclosure herein describes a process whereby 250 grams/day is achievable. In addition, the current process optionally employs the ability to crystallize lomustine thus increasing the purity and avoiding the necessity of further manipulation and transport to a facility or reactor for additional crystallization.

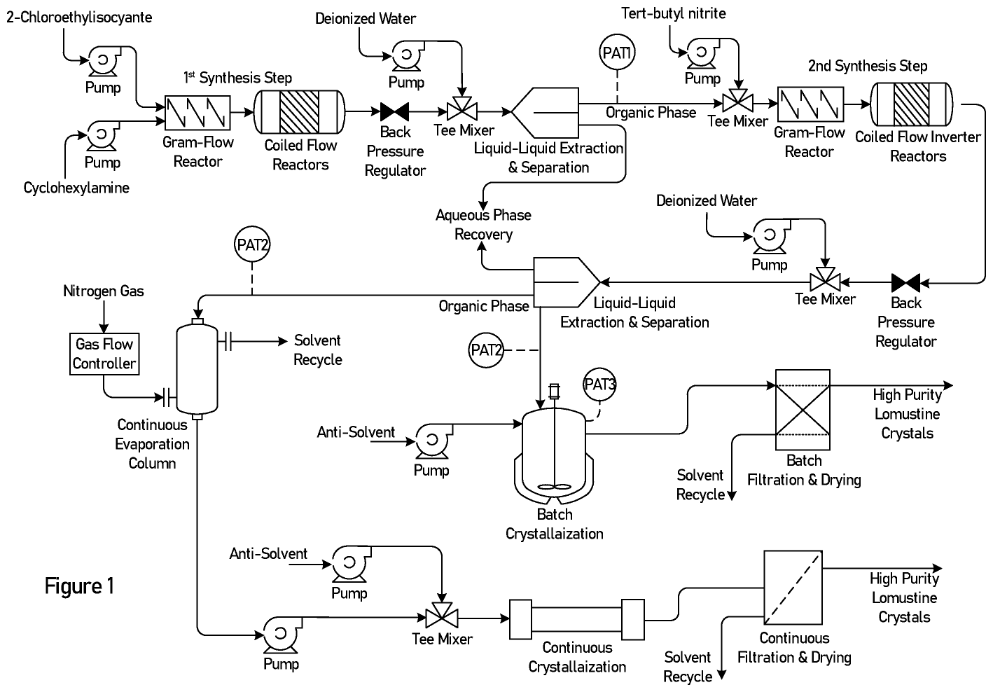

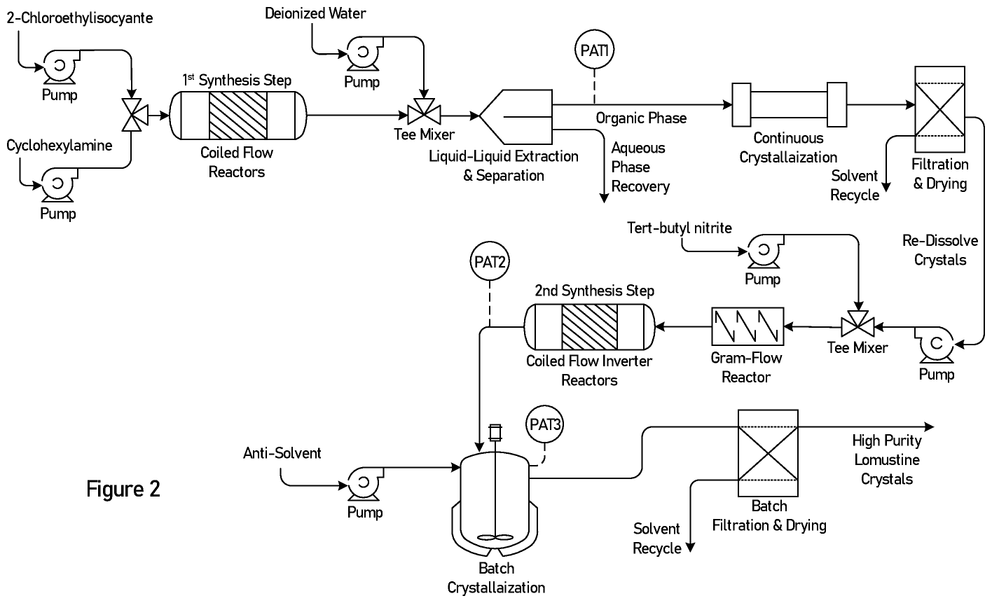

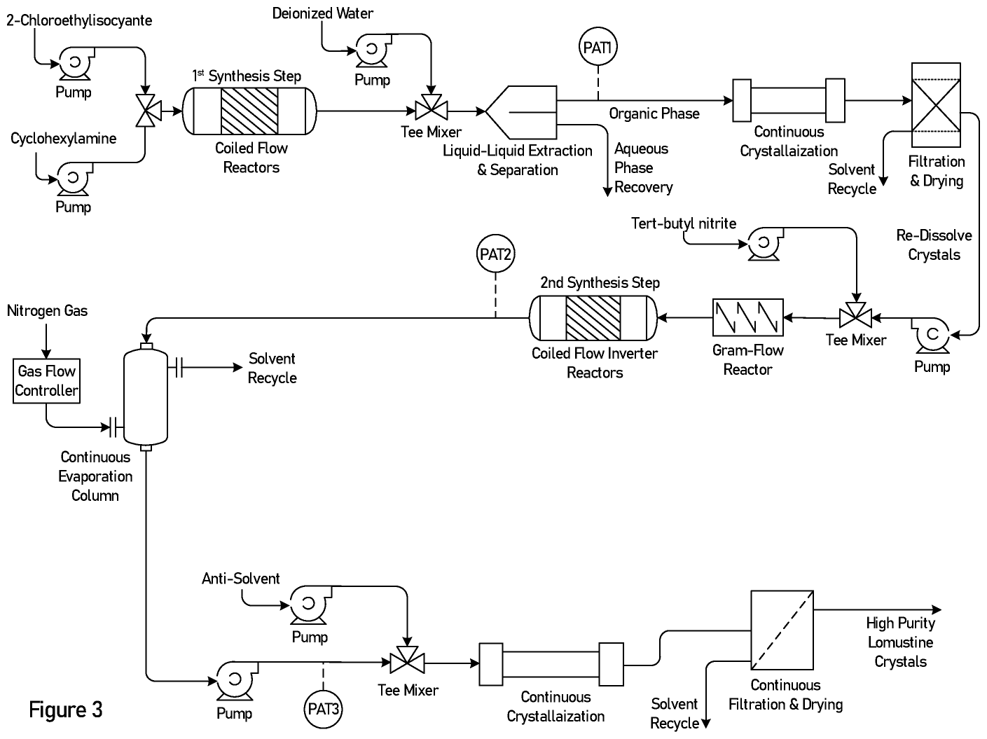

FIG. 1 describes a scale-up embodiment of the disclosure where both batch and continuous crystallization set-ups are disclosed. FIG. 2 describes a scale-up embodiment with batch crystallization and FIG. 3 describes a scale-up embodiment with continuous crystallization.

In order to create solutions for the preparation of lomustine, suitable solvents for 2-chloroethylisocyanate and cyclohexylamine are provided. Such solvents are often high boiling and immiscible with water. An example of such a suitable solvent is 2-methyltetrahydrofuran. While the solvent for 2-chloroethylisocyanate need not be the same as for cyclohexylamine, in many embodiments the solvent is the same.

When the solutions of 2-chloroethylisocyanate and cyclohexylamine are combined into a combined solution and form the intermediate 2-chloroethyl cyclohexylurea, they are flowed into a reactor. The reactor can be, for example, a continuous plug flow reactor. An example of such a reactor is a “GramFlow” reactor made by Chemtrix, Ltd. The GramFlow reactor has highly enhanced mixing due to is zig-zag flow field structural design and its integral heat exchanging plates that allow for excellent heat transfer. The GramFlow reactor has two inlets and one outlet, accommodating 1/16″ or ⅛″ tubes using ¼-28 flat bottom flangeless fittings. The reactor has a volume of 1.6 mL, a temperature range of −20 to 150 ° C., and can tolerate operating pressure up to 20 bar. All wetted materials are made of highly chemically resistant materials such as borosilicate glass, polytetrafluoroethylene, and perfluoroelastomer.

The combined solution may further be processed with a coiled flow reactor or a second GramFlow reactor. A coiled flow reactor is a reactor where the reagents are subjected to non-laminar flow, thereby improving mixing and uniformity of reaction in the flow field. To assist in the identification of process reaction completion and purity, one or more analytical instruments may be used during the process to monitor the preparation of lomustine. Examples of such instruments include Raman spectroscopy and ultraviolet-visible spectroscopy.

The reaction to prepare lomustine involves combining an aqueous phase and organic phase during the reaction processes. The aqueous phase may be introduced with the addition of t-butyl nitrite in water. Lomustine exhibits higher solubility in organic solvents (e.g., 2-methyltetrahydrofuran) than it does in water such that an extraction whereby the aqueous phase is discarded or recycled increases the purity of lomustine in the organic phase. Repeated extractions of the aqueous phase may be used to increase the yield of lomustine provided.

The continuous flow of reaction products and lomustine in solution is maintained by pumps. Previously, syringes were used to maintain a flow, but this is not well-suited for scaled-up manufacturing. The continuous flow pumps are often positive displacements units. Examples of such pumps use four or more pistons to supply continuous flow with a minimum of flow oscillation.

As shown in FIG. 1, a batch or continuous crystallization may optionally be used to prepare crystalline lomustine. In a batch crystallization process as shown in FIG. 2, an anti-solvent may be pumped into a batch crystallization reactor and combined with a solution of lomustine. The resulting mixture is then transferred into an apparatus for filtering or drying whereby crystalline lomustine is isolated. Analytical instruments, represented by “PAT 3” may be added to monitor various reaction parameters such as purity or crystallization.

As shown in FIG. 3, in an alternative crystallization process, the crystallization of lomustine occurs via continuous crystallization as opposed to batch crystallization. In this method, a solution of lomustine is transferred into a continuous evaporation column where it is concentrated and then pumped into a continuous crystallization apparatus. From there it is filtered and dried to afford crystals of lomustine.

In addition, any of the embodiments described in the following clause list are considered to be part of the invention.

Clause 1. A process for making lomustine is provided comprising:

(i) treating solutions of 2-chloroethylisocyanate with a solution of cyclohexylamine with continuous-flow pumps in a Gram-Flow flow reactor to form a combined solution,

(ii) adding deionized water with a continuous flow-pump to the combined solution to form a liquid-organic phase solution,

(iii) extracting the organic phase from the solution, and

(iv) treating the organic phase with a solution of t-butyl nitrite with a continuous flow pump in a flow reactor to form a lomustine solution.

Clause 2. The process of clause 1 wherein the combined solution is pumped into a coiled flow reactor.

Clause 3. The process of clauses 1 or 2 wherein the organic phase in step (iv) is pumped into a coiled flow reactor.

Clause 4. The process of clauses 1, 2, or 3 wherein the process is monitored by one or more analytical instruments.

Clause 5. The process of clause 4, wherein at least one of the one or more analytical instruments is a spectrometer.

Clause 6. The process of clause 5, wherein the spectrometer is a Raman spectrometer.

Clause 7. The process of clause 5, wherein the spectrometer is an ultraviolet visible spectrometer.

Clause 8. The process of clauses 1-7, further comprising an additional extraction of the organic phase with water to further purify the lomustine solution.

Clause 9. The process of clause 8, wherein the water is deionized water and is delivered via a pump.

Clause 10. The process of clauses 8-9, wherein the extraction occurs after step (iv).

Clause 11. The process of clauses 1-10, further comprising crystallizing lomustine.

Clause 12. The process of clause 11, wherein the crystallization of lomustine occurs through batch crystallization.

Clause 13. The process of clause 12, wherein the lomustine solution is combined with an anti-solvent via a pump.

Clause 14. The process of clause 13, wherein the anti-solvent is combined with the lomustine solution into a batch crystallization apparatus.

Clause 15. The process of clause 14, wherein the solvent is removed to make crystals of lomustine.

Clause 16. The process of clause 14, wherein the solvent is removed by drying. Clause 17. The process of clauses 11-16 wherein the process is monitored by one or more analytical instruments.

Clause 18. The process of clause 17, wherein at least one analytical instrument is a Raman spectrometer.

Clause 19. The process of clause 17, wherein at least one analytical instrument is an x-ray powder diffractometer.

Clause 20. The process of clause 11, wherein the crystallization of lomustine occurs through continuous crystallization.

Clause 21. The process of clause 20, wherein the lomustine solution is pumped through a continuous evaporation column to form a concentrated solution of lomustine.

Clause 22. The process of clause 21, wherein the concentrated solution of lomustine is combined with an anti-solvent from a pump into a continuous crystallization apparatus.

Clause 23. The process of clause 22, wherein the lomustine in the continuous crystallization apparatus filtered.

Clause 24. The process of clauses 23 and 24 wherein the lomustine is dried to make crystals of lomustine.

Clause 25. The process of clauses 1-24 wherein the solutions of cyclohexylamine and 2-choloroethylisocyanate are dissolved in solvents which are immiscible with water.

Clause 26. The process of clause 25, wherein the solvent is 2-methyltetrahydrofuran.

Clause 27. The process of clauses 1-6, wherein the t-butyl nitrite is in a solvent that is soluble in water.

Clause 28. The process of clause 27, wherein the solvent is THF.

Clause 29. The process of clauses 1-28, wherein at least one flow reactor is ceramic.

Clause 30. The process of clause 29, wherein the ceramic is SiC.

Clause 31. An apparatus substantially the same as FIG. 2.

Clause 32. An apparatus substantially the same as FIG. 3.

Clause 33. The process of clauses 2-30, wherein the combined solution contains 2-chloroethyl cyclohexylurea.

Clause 34. The process of clause 33, wherein the organic phase contains 2-chloroethyl cyclohexylurea.

### PROPHETIC EXAMPLE

Disclosed herein is am embodiment within the scope of the disclosure.

### Pumps

All liquid feeds to the reactors are managed using a MilliGAT MG-2-CER-XT FSPS-6 pump system (Global FIA, Fox Island, Wash.). These MilliGAT pumps are positive displacement units that utilize four coordinated pistons to supply continuous flow with minimum flow oscillation. They also offer high chemical resistance since all the wetted materials are made from PTFE and ceramic zirconia. This model has a flowrate range of 0.0024-30 mL/min and a maximum operation pressure of 200 PSI. The pump station is equipped with a PID temperature control allowing direct control of heating or cooling units in the process using a touch tablet FLUMI interface or through a customized Labview user interface.

### Heated Coiled Tubing Reactor

A customized heated coiled tubing reactor using 1/16″ inner diameter and a ⅛″ outer diameter polytetrafluoroethylene (PTFE) tubing (W. W. Grainger Inc., USA) is used. The tube itself is coiled around an engraved steel central core that contains the heating element which is controlled with an Omega CNi series PID temperature controller. The core, mounted with the coiled tubing, is clamped between two steel shells. Furthermore, the core and the shells are then placed on an enclosure containing calcium silicate insulation panels for stabilizing and maintaining the reactor set temperature. The reactor has one inlet and one outlet; thus, T-mixers or cross-mixers are used to combine multiple solutions at the reactor inlet. Flat bottom flangeless fittings and connections (¼-28) are used for connecting the tubes to the T-mixers or cross-mixers after assembling the reactor. The volume of this reactor is approximately 11 mL (tube length=5.56 cm). The maximum operating temperature for the reactor is 200 ° C. and the maximum operating pressure depends on the tube being used inside the reactor (approximately 290 PSI for ⅛″ PTFE tubing at 22.8 ° C.).

### CFI Reactor/Mixer

The second reactor to be used is a custom built coiled flow inverter (CFI) reactor. This reactor also uses ⅛″ PTFE tubing (W. W. Grainger, USA). The tubing is coiled tightly around four standard plumbing ¼″ 90° copper joints, allowing the construction square reactors. The CFI reactors offer enhanced mixing, mass transfer, and heat transfer compared to simple coiled reactors. The volume of each reactor was 8 mL (tube length=405 cm). Multiple 8 mL CFI reactors were built to enable facile changes in reaction volume or residence time by connecting the desired number of CFI units in series using ¼-28 flat bottom flangeless fittings and unions.

### GramFlow Reactor/Mixer

The GramFlow reactor (Chemtrix, Ltd., Netherlands) is a continuous plug flow reactor with highly enhanced mixing due to is zig-zag flow field structural design and its integral heat exchanging plates that allow for excellent heat transfer. The GramFlow reactor has two inlets and one outlet, accommodating 1/16″ or ⅛″ tubes using ¼-28 flat bottom flangeless fittings. The reactor has a volume of 1.6 mL, a temperature range of −20 to 150 ° C., and can tolerate operating pressure up to 20 bar. All wetted materials are made of highly chemically resistant materials such as borosilicate glass, PTFE, and FFKM.

### Liquid-Liquid Separators

Two SEP-10 units (Zaiput Flow Technologies, USA) are used for liquid-liquid separations. The SEP-10 unit utilizes a porous hydrophobic PTFE membrane (OB-400) allowing the organic phase (the wetting phase) to flow through the membrane while the aqueous phase passes over the membrane and out of the separator. The SEP-10 unit has an internal pressure controller that maintains the pressure differential across the membrane to enable better separation of the two phases. The separation efficiency of this unit depends on multiple factors including, but not limited to, membrane material and pore size, the total inlet flowrate, separation temperature, and the interfacial tension values between the organic and the aqueous phase.

### Synthesis of Lomustine

Solutions of cyclohexylamine and 2-chloroethyl isocyanate are prepared in anhydrous 2-methyltetrahydrofuran separately under a dry N₂ atmosphere. The solutions are added to two amber GL45 glass bottles to protect them from light (2-chloroethyl isocyanate is light sensitive). All the transfer lines ( 1/16″ inner diameter and ⅛″ outer diameter PTFE tubing) for 2-chloroethyl isocyanate throughout the process are covered with aluminum-tape for light protection. The transfer lines were placed in the charged amber starting material bottles after installing 0.2 μm PTFE inlet filters before passing them through GL45 solvent delivery caps and connecting them to the inlets of the milliGAT pump array. The solvent delivery caps have two ports, one for the transfer line and the other for N₂ flow since the bottles are kept under inert conditions as the solvent is dispensed. Following the pump outlet, a T-relief valve assembly followed by a check valve is installed on the outlet of each pump before connecting the transfer tubes to the T-mixer ahead of the heated coiled tubing reactor. The T-relief valve assembly and the check valve are used to prevent over-pressurizing the process and avoid any backflow in the tubes. The temperature of the reactor is set to ˜50 ° C. and the residence time is 1-3 minutes. The outlet of the heated coiled tubing reactor, containing the solution of 2-chloroethyl cyclohexylurea intermediate resulting from the reaction of the cyclohexylamine and the 2-chloroethyl isocyanate, is connected to a T-mixer where deionized water is added in order to extract water-soluble impurities while retaining the 2-chloroethyl cyclohexylurea intermediate in the organic phase before entry of the mixture into the Zaiput membrane separator. The organic phase outlet of the separator is connected to the next reaction step and the other outlet transported the aqueous extraction phase to a waste container. A solution of tert-butyl nitrite is prepared in anhydrous tetrahydrofuran under dry N₂, placed in an amber GL45 glass bottle, and connected to a pump following the same procedure as described above. Using another MilliGAT pump, the tert-butyl nitrite is added to the organic phase containing the 2-chloroethyl cyclohexylurea intermediate through a T-mixer. The reaction mixture is then passed through a series of thirteen CFI reactors at 20° C. with a total residence time of 10 minutes to generate lomustine. The outlet of the CFI reactor train containing the lomustine product is connected to a T-mixer where deionized water is added in order to extract water-soluble impurities. After flowing through the Zaiput membrane separator, the organic phase is retained for further purification of the lomustine product via crystallization while the other outlet transfers the aqueous phase to a waste container.

1. A process for making lomustine is provided comprising:
(i) treating a solution of 2-chloroethylisocyanate with a solution of cyclohexylamine with continuous-flow pumps in flow reactors to form a combined solution,
(ii) adding deionized water with a continuous flow-pump to the combined solution to form a liquid-organic phase solution,
(iii) extracting the organic phase from the solution, and
(iv) treating the organic phase with a solution of t-butyl nitrite with a continuous flow pump in a flow reactor to form a lomustine solution.

2. The process of claim 1, wherein the combined solution is pumped into a coiled flow reactor.

3. The process of claim 1, wherein the organic phase in step (iv) is pumped into a coiled flow reactor.

4. The process of claim 1, wherein the process is monitored by one or more analytical instruments.

5. The process of claim 4, wherein at least one of the one or more analytical instruments is a spectrometer.

6. The process of claim 5, wherein the spectrometer is a Raman spectrometer.

7. The process of claim 5, wherein the spectrometer is an ultraviolet visible spectrometer.

8. The process of claims 1, further comprising an additional extraction of the organic phase with water to further purify the lomustine solution.

9. The process of claim 8, wherein the water is deionized water and is delivered via a pump.

10. The process of claims 8, wherein the extraction occurs after step (iv).

11. The process of claims 1, further comprising crystallizing lomustine solution.

12. The process of claim 11, wherein the crystallization of lomustine solution occurs through batch crystallization.

13. The process of claim 12, wherein the lomustine solution is combined with an anti-solvent via a pump.

14. The process of claim 13, wherein the anti-solvent is combined with the lomustine solution into a batch crystallization apparatus.

15. The process of claim 14, wherein the solvent is removed to make crystals of lomustine.

16. The process of claim 14, wherein the solvent is removed by drying.

17. The process of claims 11, wherein the crystallization is monitored by one or more analytical instruments.

18. The process of claim 17, wherein at least one analytical instrument is a Raman spectrometer.

19. The process of claim 17, wherein at least one analytical instrument is an x-ray powder diffractometer.

20. The process of claim 11, wherein the crystallization of lomustine occurs through continuous crystallization.
